(19) United States
(12) Patent Application Publication
(10) Pub. No.: US 2017/0080038 A1
(43) Pub. Date: Mar. 23, 2017

(54) TRANSDERMAL THERAPEUTIC SYSTEM CONTAINING LAVENDER OIL

(71) Applicant: LTS Lohmann Therapie-Systeme AG, Andemach (DE)

(72) Inventors: Petra BOTZEM, Andernach (DE); Thomas HILLE, Neuwied (DE)

(21) Appl. No.: 15/312,087

(22) PCT Filed: May 8, 2015
(86) PCT No.: PCT/EP2015/000955

§ 371 Date: Nov. 17, 2016

(30) Foreign Application Priority Data
May 20, 2014 (EP) 14169037.0

(51) Int. Cl. A61K 36/53; A61M 37/00; A61F 13/02; A61K 9/70

(52) U.S. Cl. CPC A61K 36/53; A61K 9/7084; A61M 37/00; A61F 13/0246; A61M 2207/10

(57) ABSTRACT

The invention relates to transdermal therapeutic systems (TTS) containing lavender oil as active substance and to their use in the prevention and/or treatment of excitation, sleep disturbances, anxiety and nervousness, in particular of excitation in anxiety depression. The invention also relates to methods for producing these TTS, in which methods said TTS are produced using support materials from fibrous components and are charged with active substance by as printing method.

The invention relates to a transdermal therapeutic system (TTS) containing the active ingredient lavender oil and to its use in the prophylaxis and/or treatment of restlessness, sleep disorders, anxieties and nervousness, especially of restlessness due to an anxious mood.

The invention also relates to methods for producing said transdermaI therapeutic systems, in which the systems are produced using support materials composed of fibrous constituents and are loaded with active ingredient by means of a printing process.

US 2008/124410 A1 discloses the prophylactic and therapeutic use of lavender oil for the treatment of neurasthenia, somatization disorders and other stress-associated diseases and also lavender oil-containing medicaments and dietetic food products and also preparations and capsules as oral administration forms.

However, when lavender oil is administered orally, there may occasionally be occurrences of nausea and of an eructation and a lavender oil mouth odor.

Owing to their possibly short half-life and their first-pass effect, the oral administration of active ingredients may be problematic. In this connection, a short half-life would necessitate a frequent intake of the substance and a high first-pass effect a high dosage. Whereas the intake frequency may possibly be overcome by an appropriate oral formulation, the problem of a high first-pass effect can in principle only be solved by a nonoral intake of the active ingredient.

It is an object of the present invention to provide a means which can be used in an effective manner for the prophylaxis and/or treatment of restlessness, sleep disorders, anxieties and nervousness, which does not exhibit the disadvantages mentioned and which is largely free of adverse effects.

This object is achieved by a transdermal therapeutic system (TTS) containing the active ingredient lavender oil.

Transdermal therapeutic systems are systems for the controlled administration of active pharmaceutical ingredients across the skin. They have been used for quite some time for treating different diseases, physical and mental dysfunctions, complaints and also indispositions. Transdermal therapeutic systems are layered products in the form of plasters which comprise an active-ingredient-impermeable backing layer, at least one active-ingredient-containing reservoir or matrix layer, optionally one membrane controlling the rate of the release of active ingredient, and a detachable protective layer which is removed from the TTS before use thereof.

A transdermal therapeutic system is provided with a pressure-sensitively adhering layer for fastening the TTS on the skin and also for ensuring the controlled administration of the active ingredient. Said pressure-sensitively adhering layer can be identical to the active-ingredient-containing matrix layer or the skin-side active-ingredient-containing layer, but can also be additionally present if the (skin-side) active-ingredient-containing layer or the optionally present membrane is not pressure-sensitively adhering.

The backing layer of a TTS must be impermeable for the active ingredient present in the TTS in order to prevent an undesired escape of the active ingredient from the side of the TTS that is facing away from the skin. To this end, use is made in particular of metal foils, specific plastics films and also composite laminates of said materials. Composite laminates composed of aluminum and plastics materials such as polyethylene terephthalate are most common. The advantage of said composite laminates is that aluminum foils can be produced cost-effectively and are impermeable with respect to virtually all active pharmaceutical ingredients. Moreover, aluminum foils are light-impermeable, providing the advantage of a reliable protection against light specifically for light-sensitive active ingredients.

WO 2007/006529 A1 describes a TTS for delivering at least one active pharmaceutical ingredient, which TTS comprises at least one enclosed, preferably encapsulated, fragrance, such as inter alia lavender oil, which fragrance is released no later than when the system is applied. The fragrance serves to improve patient acceptance in relation to the use of the TTS, but not to systemically administer lavender oil.

The invention provides transdermal therapeutic systems (TTSs) comprising
a) a backing layer which is facing away from the skin and is impermeable for the active ingredient,
b) an active-ingredient depot containing the active ingredient lavender oil,
c) a matrix which is in contact with the active-ingredient depot and controls the delivery of the active ingredient,
d) a pressure-sensitively adhering fixing means for the therapeutic system on the skin and
e) a detachable protective layer, the active-ingredient depot additionally containing support material comprising fibrous constituents.

True lavender or narrow-leaved lavender (Lavandula angustifolia, also known as Lavandula officinalis, Lavandula vera) is a plant species from the labiate family (Lamiaceae). The plant is mainly used as an ornamental plant or for obtaining fragrances. The distribution area extends from the Canary islands through the entire Mediterranean area to the Indian subcontinent. Especially essential oils (lavender oil) in the aboveground parts, and also caffeic acid and depsides thereof in the leaves, have been described as ingredients. The lavender oil is composed of linalyl acetate, linalool, camphor and eucalyptol. Its ingredients are 40-50% esters, 25-35% monoterpenols, monoterpenes, sesquiterpenes, ketones and oxides. The essential oils produced from lavender are traditionally used for cosmetic products, but also for therapeutic purposes, for example in aromatherapy. For instance, antibacterial, antifungal, spasmolytic, sedative and antidepressive effects have been described for lavender oil (H. M. A. Cavanagh et al. (2002) , Phytother. Res. 16, 301-308).

Lavender oil can be produced according to known production methods, preferably by steam distillation of freshly harvested lavender flowers.

In Germany, preparations from lavender flowers in the form of tea infusions, as extract and also as bath additive for the indications restlessness, disorders relating to falling asleep, functional upper abdominal complaints, meteorism and in balneotherapy have been positively monographed (monography of commission E of the former German federal health office).

The active-ingredient depot of the TTSs according to the invention can contain lavender oil or the pure active ingredients linalool or linalyl acetate, also in combination with at least one pharmaceutically acceptable excipient. The active-ingredient content of a TTS is from 30 to 400 mg, preferably from 40 to 300 mg, more particularly from 50 to 150 mg.

The support material of the TTSs according to the invention comprises fibrous constituents in which the entities are comparatively long, thin and flexible entities composed of natural or synthetic material. The fibrous constituents composed of natural materials include, for example, plant fibers, animal fibers and also mineral fibers. Plant fibers such as raffia, cotton, hemp, coconut, linen, kapok or ramie generally consist of cellulose. The animal fibers include silk and hair (wool). A naturally occurring mineral fiber is asbestos. The synthetic fibers include fibers composed of polycaprolactam, nylon, but also artificial silk, glass fibers and carbon fibers. Multiple fibers jointly form larger structures. For instance, textile fibers can jointly form a filament, a cord or a fabric. Cellulose fibers are, for example, used for paper and textile production. A preferred support material is paper, textile material or a nonwoven.

In a preferred embodiment, the active-ingredient depot has an area of from 10 to 35 cm², preferably from 12 to 30 cm², more particularly from 15 to 25 cm².

The matrix layer and the pressure-sensitively adhering fixing means of the TTS according to the invention consist of the same material or of different materials. They comprise a material which is selected from the group consisting of pressure-sensitively adhering polymers based on acrylic acid and/or methacrylic acid and also their esters, polyacrylates, polyisobutylene, polyvinyl acetate, ethylene-vinyl acetate copolymer, natural and/or synthetic rubbers, for example acrylonitrile-butadiene rubber, butyl rubber or neoprene rubber, styrene-diene copolymers such as styrene-butadiene block copolymers and hot-melt adhesives, or which is produced on the basis of pressure-sensitively adhering silicone polymers or polysiloxanes.

Preferably, said material is selected from the group comprising cationic copolymers based on dimethylaminoethyl methacrylate and neutral methacrylic esters, for example Eudragit® E 100, and neutral copolymers based on butyl methacrylate and methyl methacrylates, for example Plastoid® B.

Suitable materials for the active-ingredient-impermeable backing layer are especially polyesters, which are distinguished by a particular strength, such as, for example, polyethylene terephthalate and polybutylene terephthalate, but additionally virtually any other skin-compatible plastics, such as polyvinyl chloride, polyurethane, polyvinylidene chloride, ethylene-vinyl acetate copolymers, polyvinyl acetate, vinyl acetate-vinyl chloride copolymers, nylon, polyethylene, polypropylene, polyurethanes, polyamide, cellulose derivatives and many more. Preference is given to polyethylene terephthalate, plasticized vinyl acetate-vinyl chloride copolymers, nylon, ethylene-vinyl acetate copolymers, plasticized polyvinyl chloride, polyurethane, polyvinylidene chloride, polypropylene, polyethylene and polyamide. In particular cases, the backing layer can be provided with an additional overlay, for example by vapor deposition with metals, more particularly aluminum.

In the case of the detachable protective layer, it is absolutely possible to use the same materials as for the backing layer, provided that they have been provided with a detachment-enabling finish by means of a suitable surface treatment, such as, for example, siliconization. However, it is also possible to use other detachable protective layers such as, for example, polytetrafluoroethylene-treated paper or Cellophane® (cellulose hydrate).

The method for producing the TTSs according to the invention is characterized in that
a laminate Composed of an active-ingredient-impermeable support layer, a pressure-sensitively adhering fixing layer and a matrix layer is produced,
a depot layer intended for the accommodation of the active ingredient is applied to the matrix side of said laminate,
individually dispensed portions of a free-flowing, active-ingredient-containing preparation are applied to said depot layer by means of a printing process,
optionally a further matrix layer is laminated thereon and
the thus obtained laminate is lastly provided with an active-ingredient-impermeable backing layer,
   it being possible to singularize the transdermal therapeutic systems from the hitherto formed composite laminate by cutting and/or punching before or after the application of the active-ingredient-containing preparation.

The above printing process can be a pad printing process. Such a process is known, from U.S. Pat. No. 5,110,599, to which full reference is made.

The above printing process can also be a process in which the active-ingredient-containing preparation is transferred to the depot layer intended for the accommodation of the active ingredient by means of an application device distribution plate provided with at least one passage. Such a process is known from U.S. Pat. No. 6,187,322, to which full reference is made.

The active ingredient lavender oil can be directly applied by means of the above two printing processes. Customarily, the active, ingredient is, however, used in the form of a liquid having the desired viscosity as a result of addition of suitable solvents and/or excipients. Suitable solvents are in principle all common organic solvents, such as, for example, ethanol, isopropyl alcohol, heptane, hexane, ethyl acetate, petroleum ether, benzine, acetone, glycerol, DEET (N,N-diethyl-3-methylbenzamide), THF and also many oils, for example silicone oil, paraffin, triglycerides, neutral oil or plant oils. Excipients which increase the viscosity of the lavender oil are polymers which are also used for producing the matrix layers. Particularly suitable is PVP or polymethacrylate. The viscosity of the active-ingredient containing preparation to be used as printing medium is preferably within the range from 10 to 100 dPa·s, particularly preferably within a range from 15 to 25 dPa·s.

The invention further provides for the use of a TTS comprising
a) a backing layer which is facing away from the skin and is impermeable for the active ingredient,
b) an active-ingredient depot containing the active ingredient lavender oil,
c) a matrix which is in contact With the active-ingredient depot and controls the delivery of the active ingredient and
d) a pressure-sensitively adhering fixing means for the therapeutic system on the skin, the active-ingredient depot additionally containing support material comprising fibrous constituents, which TTS is adhered to the skin, the active ingredient being delivered transdermally in a prophylactically or therapeutically effective quantity, preferably over a period of at least 2 hours, in the prophylaxis and/or treatment of restlessness, sleep disorders, anxieties and nervousness,
   also a method for the prophylaxis and/or treatment of restlessness, sleep disorders, anxieties and nervousness, in which method such a TTS is used.

The invention shall be elucidated below on the basis of an exemplary embodiment and the accompanying drawings, in which the structure of TTSs according to the invention is depicted schematically, without the invention being restricted thereto. In this connection:

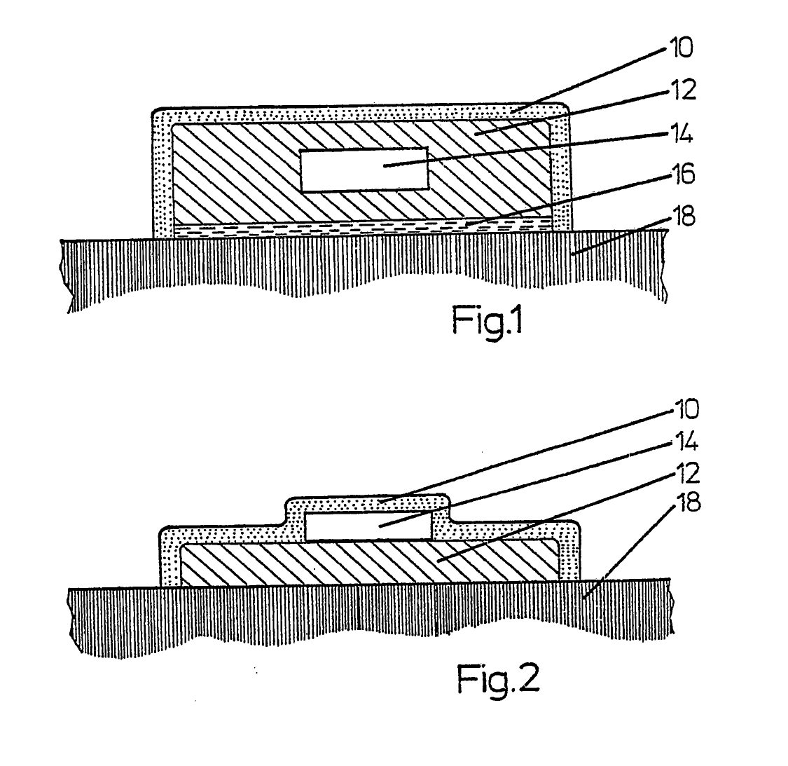

FIG. 1 shows a section through a preferred embodiment of a TTS according to the invention; and

FIG. 2 shows a section through a further preferred embodiment of a therapeutic system in which the active-ingredient depot is situated between backing layer and reservoir matrix. In said figure, the TTS is depicted as being adhered to the skin after removal of the detachable protective layer.

FIG. 1 schematically depicts a section through a therapeutic system according to the invention, which therapeutic system is fastened on the skin 18 by means of a fixing means 16. Situated on the fixing means 16 is the reservoir matrix layer 12, which is preferably free of active ingredient at the time of production (the saturation with active ingredient occurs during storage). Embedded into the reservoir matrix is an active-ingredient depot 14 which delivers the active ingredient to reservoir matrix material and then releases it to the skin 18 through the fixing means 16. The therapeutic system is outwardly enclosed by means of a backing layer 10 which is impermeable for the active ingredient and preferably also for moisture and, at the same time, exercises a support function for the system.

FIG. 2 shows a further variant, of the system according to the invention, in which variant an active-ingredient depot 14 lies on a reservoir matrix layer 12 and is covered by a backing layer 10. In this drawing, the fixing means is not depicted, since, in this case, the pressure-sensitively adhering reservoir matrix assumes the function of the fixing means. This embodiment is advantageous to the extent that its production is conceivably simple; it is merely necessary to apply defined quantities to a prefabricated matrix layer and to enclose the entire v means of a backing layer 10.

Typical thickness measurements for TTSs according to the invention are: for an overall thickness of approximately 123 μm to 5550 μm, preferably 285 μm-1550 μm; thickness of the backing layer: 8-150 μm, preferably 15-100 μm; thickness of the matrix in contact with the active-ingredient depot 100-5000 μm, preferably 200-1300 μm; thickness of the protective layer: 15-400 μm, preferably 70-150 μm. Such layer thicknesses are approximately achieved when surface coating weights for the matrix of 100-5000 g/m² are selected.

### EXAMPLE

A pressure-sensitive adhesive HS is first produced by homogenization of
a) 933 g of a commercial product (Duro-Tak® 387-2518 from Henkel, Dusseldorf, Germany—this is a 40% solution of a self-crosslinking acrylate polymer based on 2-ethylhexyl acrylate, vinyl acetate, hydroxyethyl acrylate, glycidyl methacrylate and titanium chelate ester in a solvent mixture composed of ethyl acetate, ethanol, heptane and methanol) with
b) 8 g of a triglyceride of fractionated coconut fatty acids (C₈-C₁₀; Miglyol® 812 from Evonik Witten, Germany).

In addition, 6210 g of Duro-Tak® 387-2516, 553 g of ethyl acetate and 311 g of ethanol are admixed with 66 g of the abovementioned triglyeride and also 626 g of an acrylic resin composed of dimethylaminoethyl methacrylate and neutral methacrylic esters Eudragit® E 100 from Röhm-Pharma, Darmstadt Germany) and homogenized (adhesive MS).

In addition, 72 g of Eaudragit® E 100 are introduced into 100 g of lavender oil and dissolved therein. The result is the active-ingredient preparation.

The pressure-sensitive adhesive HS is applied to an abhesively finished protective layer (A) such that a layer of pressure-sensitive adhesive having a surface weight of 40 g/m² is formed after evaporation of the solvents.

The adhesive MS is applied to another abhesively finished protective layer (B) such that a film having a surface weight of 220 g/m² is formed after evaporation of the solvents. Said film is laminated onto the layer of pressure-sensitive adhesive applied to the protective layer (A). The result is the bottom sheet.

In a further coating action, the adhesive MS is applied to a further abhesively finished protective layer (C) such that a film having a surface weight of 110 g/m² is formed after evaporation of the solvents, onto which film the backing layer impermeable for the active ingredient is laminated. In this case, the top sheet is formed.

After removal of the abhesively finished protective layer (B) from the bottom sheet, circular blanks composed of tea filter paper (surface weight 40 g/m²) are positioned centrally.

Thereafter, the active-ingredient preparation is printed onto the nonwoven circular blanks by means of an egg-shaped silicone sponge rubber pad with a Shore hardness of 6. The quantity of the active-ingredient preparation is measured such that each TTS later contains 180 mg of lavender oil.

After removal of the adhesively finished protective layer (C), the top sheet is laminated onto the bottom sheet (equipped with nonwoven circular blanks and doped with active-ingredient preparation), and TTSs are punched out.

1. A transdermal therapeutic system (TTS) comprising a backing layer which is facing away from the skin and is impermeable for an active ingredient,
a) an active-ingredient depot containing lavender oil as the active ingredient or linalool or linalyl acetate as pure active ingredients,
b) a matrix which is in contact with the active-ingredient depot and controls the delivery of the active ingredient,
c) a pressure-sensitively adhering fixing means for the therapeutic system on skin and
d) a detachable protective layer,
wherein the active-ingredient content is from 30 to 400 mg and the active-ingredient depot additionally contains support material comprising fibrous constituents.

2. The TTS as claimed in claim 1, wherein the active-ingredient depot contains the pure active ingredient.

3. The TTS as claimed in claim 1, wherein the active-ingredient depot contains at least one pharmaceutically acceptable excipient.

4. The TTS as claimed in claim 1, containing from 30 to 400 mg of lavender oil as active ingredient or the pure active ingredients linalool or linalyl acetate.

5. The TTS as claimed in claim 1, wherein the support material is paper, textile material or a nonwoven.

6. The TTS as claimed in claim 1, wherein the active-ingredient depot has an area of from 10 to 35 cm₂.

7. The TTS as claimed in claim 1, wherein the backing layer consists of a material selected from polyethylene terephthalate, plasticized vinyl acetate-vinyl chloride copolymers, nylon, ethylene-vinyl acetate copolymers, plasticized polyvinyl chloride, polyurethane, polyvinylidene chloride, polypropylene, polyethylene or polyamide.

8. The TTS as claimed in claim 1, wherein the matrix and the pressure-sensitively adhering fixing means are identical or different and comprise a material which is selected from the group consisting of pressure-sensitively adhering polymers based on acrylic acid and/or methacrylic acid and also their esters, polyacrylates, polyisebutylene, polyvinyl acetate, ethylene-vinyl acetate copolymer, natural and/or synthetic rubbers, styrene-diene copolymers and hot-melt adhesives, or which is produced on the basis of pressure-sensitively adhering silicone polymers or polysiloxanes.

9. The TTS as claimed in claim 1, wherein the matrix and the pressure-sensitively adhering fixing means are identical or different and comprise a material selected from the group consisting of cationic copolymers based on dimethylaminoethyl methacrylate and neutral methacrylic esters and neutral copolymers based on butyl methacrylate and methyl methaerylates.

10. A method for producing a TTS as claimed in claim 1, said method comprising:
producing a laminate composed of an active-ingredient-impermeable support layer, a pressure-sensitively adhering fixing layer and a matrix layer,
applying a depot layer for the accommodation of the active ingredient to the matrix side of said laminate,
printing individually dispensed portions of a free-flowing, active-ingredient-containing preparation onto said depot layer by means of a printing process,
optionally laminating a further matrix layer thereon and providing the thus obtained laminate with an active-ingredient-impermeable backing layer,
said method further comprising singularizing the transdermal therapeutic systems from the hitherto formed composite laminate by cutting and/or punching before or after the application of the active-ingredient-containing preparation.

11. The method as claimed in claim 10, wherein the printing process is a pad printing process.

12. The method as claimed in claim 10, wherein the printing process comprises transferring the active-ingredient-containing preparation onto the depot layer intended for the accommodation of the active ingredient by means of an application device distributor plate provided with at least one aperture.

13. The method as claimed in claim 10, wherein said method further comprises adjusting the viscosity of the free-flowing, active-ingredient-containing preparation by adding suitable solvents and/or excipients.

14. (canceled)

15. A method for the prophylaxis and/or treatment of restlessness, sleep disorders, anxieties and nervousness comprising adhering a TTS as claimed in claim 1 to skin, said TTS comprising
a) a backing layer which is facing away from the skin and is impermeable for the active ingredient,
b) an active-ingredient depot containing the active ingredient lavender oil or the pure active ingredients linalool or linalyl acetate,
c) a matrix which is in contact with the active-ingredient depot and controls the delivery of the active ingredient and
d) a pressure-sensitively adhering fixing means for the therapeutic system on the skin, the active-ingredient content being from 30 to 400 mg and the active-ingredient depot additionally containing support material comprising fibrous constituents,
said method delivering the active ingredient transdermally in a prophylactically or therapeutically effective quantity.

16. (canceled)

17. The TTS as claimed in claim 4, wherein said TTS contains from 40 to 300 mg of lavender oil as active ingredient or the pure active ingredients linalool or linalyl acetate.

18. The TTS as claimed in claim 4, wherein said TTS contains from 50 to 150 mg of lavender oil as active ingredient or the pure active ingredients linalool or linalyl acetate.

19. The TTS as claimed in claim 6, wherein the active-ingredient depot has an area of from 12 to 30 cm².

20. The TTS as claimed in claim 6, wherein the active-ingredient depot has an area of from 15 to 25 cm².

21. The TTS as claimed in claim 8, natural and/or synthetic rubbers are acrylonitrile-butadiene rubber, butyl rubber or neoprene rubber and the styrene-diene copolymers are styrene-butadiene block copolymers.

22. A method for the prophylaxis and/or treatment of restlessness, sleep disorders, anxieties and nervousness as claimed in claim 15, wherein said method is performed over a period of at least 2 hours and up to 48 hours.
